(19) United States
(12) Patent Application Publication
(10) Pub. No.: US 2025/0318732 A1
(43) Pub. Date: Oct. 16, 2025

(54) PROBE ASSEMBLY AND METHOD OF MANUFACTURE

(71) Applicant: The Regents of the University of Michigan, Ann Arbor, MI (US)

(72) Inventors: Eunah Ko, Ann Arbor, MI (US); Euisik Yoon, Superior Township, MI (US)

(21) Appl. No.: 18/862,940

(22) PCT Filed: May 1, 2023
(86) PCT No.: PCT/US2023/020633

§ 371 Date: Nov. 4, 2024

Related U.S. Application Data
(60) Provisional application No. 63/338,426, filed on May 4, 2022.

(51) Int. Cl. A61B 5/00; A61B 5/294; A61L 31/06; A61N 1/05; A61N 5/06

(52) U.S. Cl. CPC A61B 5/0084; A61B 5/294; A61L 31/06; A61N 1/0553; A61N 5/0601; A61B 2562/0233; A61B 2562/125; A61N 2005/0612

(57) ABSTRACT

A probe assembly comprising a probe shank having a plurality of polymer layers. The plurality of polymer layers includes a first polymer layer and a second polymer layer. The first polymer layer and the second polymer layer sandwich one or more recording traces or one or more stimulating traces such that the first polymer layer, the second polymer layer, the one or more recording traces, and the one or more stimulating traces are configured to conform around an anatomical structure. A method of manufacturing involves treating the first polymer layer to increase a surface area on an adhesion portion of the first polymer layer. Another method of manufacturing involves monolithically fabricating a light emitting diode (LED) that is equal to or less than 500 square microns, and applying a polyimide layer to form a probe shank around the LED.

This invention was made with government support under NS113283 awarded by the National Institutes of Health. The government has certain rights in the invention.

### FIELD

This invention relates generally to probe assemblies, and more particularly, to neural optoelectrode probe assemblies and methods of manufacture.

### BACKGROUND

Understanding complex neuronal circuitry and its functions requires a specialized tool which is capable of (i) recording local field potential variation, (ii) manipulating membrane voltage potential variations, while at the same time (iii) stably functioning for a long period without significant tissue degeneration or device migration. With a typical more rigid silicon shank, these goals, particularly with respect to long-term functionality and placement of the probe, may be difficult to accomplish.

The brain is a complex organ which makes it not easy to understand how it is exactly operating by simply dissecting and looking into it. Thus, it takes time and effort for the development of a direct neural interface. However, to properly function inside the brain for a long time and discern the neuronal circuitry, there are several challenges that must be overcome. First, the physical size of the probe (including the insertion method) must be small enough to prevent potentially severe tissue damage, otherwise the targeted region of interest can be injured and neuron activity cannot be adequately interpreted (minimally-invasive structure required). This also hints that the material that is composing such a probe should not evoke some harsh tissue reaction. Second, the probe should be equipped with some form of neuron circuit manipulating mode (so that a specific circuitry unit can be interpreted cell-by-cell by controlling the cell activity artificially), while having enough spatiotemporal resolution. Lastly, in order to comprehend the long-term memory or behavior activity, the interfacing probe must survive for a sufficiently long duration inside the brain region of interest.

One of the promising candidates meeting these requirements includes inorganic LED (ILED) integrated optogenetic-based selective cell protein stimulation, which stimulates genetically modified opsin (such as gene expressed channelrhodopsin-2) upon illumination of LED light. In contrast to other types of optical power delivering methods such as waveguide-based light delivery devices, ILED based probes have strong advantages such as their scalability (high spatial resolution), fabrication process compatibility (mass-production by utilizing wafer-level manufacturing), and high temporal resolution. In 2015, Fan Wu et al. demonstrated the first ILED integrated neural probe shaped as a traditional Michigan-type equipped with a total of 12 micro-sized GaN ILEDs and 32 recording sites. The size of the unit LED was kept similar to the size of the neuron soma, and each of the LEDs was operated independently from others, realizing single-cell status modulation and recording Furthermore, the span covered by the probe (for both stimulation and recording sites) was enlarged through microfabrication development (Kim et al., bioRxiv 2020). This large-scale device enabled the simultaneous neuron modulation/readout at greater than an 8 times wider area versus the former version of the Michigan probe, while maintaining comparable functionalities. Yet, even these advanced technologies still could not go over the last—but not least—hurdle: long-term chronic functionality. To construe and portray the neuronal circuitry, an apparatus that allows us to explore deep-brain for a sufficiently continued amount of time is essential, and this is where the neuroscience field demands a flexible instrument: the actual neural interfacing part of the probe should be soft enough, meaning the local brain stiffness must be reduced by means of reduced Young's modulus of the material. The local brain stiffness is defined as Ewt³/4L³, where E is the Young's modulus, w is the width of the probe shank, t is the thickness, and L is the total length of the probe shank. Substituting the rigid or semi-rigid substrate that holds all the components of the neural interface to a softer, more flexible probe shank can help alleviate tissue reaction and thereby can reduce the possibility of the probe function deterioration. Also, the location of the inserted probe can remain in the inserted position with respect to the anatomical structure during the whole chronic experiment term.

### SUMMARY

According to one embodiment, there is provided a probe assembly comprising a probe shank having a plurality of polymer layers. The plurality of polymer layers includes a first polymer layer and a second polymer layer. The first polymer layer and the second polymer layer sandwich one or more recording traces or one or more stimulating traces such that the first polymer layer, the second polymer layer, the one or more recording traces, and the one or more stimulating traces are configured to conform around an anatomical structure.

In some embodiments, the plurality of polymer layers is made from a polyimide-based material.

In some embodiments, the plurality of polymer layers is made from a parylene-based material, a PDMS-based material, or a silicone-based material.

In some embodiments, one or more polymer layers of the plurality of polymer layers includes a plurality of hills and valleys.

In some embodiments, the probe shank comprises a stimulating probe having the one or more stimulating traces stacked against a recording probe having the one or more recording traces.

In accordance with one embodiment, there is provided a probe assembly comprising a recording probe having one or more recording traces located between a plurality of polymer layers, and a stimulating probe having one or more stimulating traces located between a plurality of polymer layers. The stimulating probe is stacked against the recording probe.

In some embodiments, the recording probe includes an optical window configured to at least partially expose a light source on the stimulating probe.

In some embodiments, the light source on the stimulating probe is an inorganic light emitting device (ILED), an organic light emitting device (OLED), a quantum dot (QD), or an electroluminescent device.

In some embodiments, there are a plurality of light sources, wherein one or more light sources of the plurality of light sources are different colors having different wavelengths.

In some embodiments, a metal shielding layer is placed between the recording probe and the stimulating probe.

In some embodiments, various functional subassemblies are stacked together, the various functional assemblies including one or more temperature sensors, one or more neurotransmitter sensors, and/or one or more microfluidic layers or channels.

In some embodiments, an interposer is connected between a cable and the recording probe and the stimulating probe to minimize micromotion of a headstage.

In some embodiments, a circuit chip is hybrid-integrated in the interposer to change the signal-to-noise ratio of recorded signals and reduce a number of traces by digitally multiplexing stimulation signals and the recording signals.

In accordance with one embodiment, there is provided a method of manufacturing a probe assembly comprising the steps of treating a first polymer layer to increase a surface area on an adhesion portion of the first polymer layer, and adhering the first polymer layer to a second polymer layer. The adhesion portion of the first polymer layer contacts the second polymer layer.

In accordance with one embodiment, there is provided a method of manufacturing a probe assembly comprising the steps of monolithically fabricating a light emitting diode (LED) that is equal to or less than 500 square microns, and applying a polyimide layer to form a probe shank around the LED.

In some embodiments, there is a step of temporarily attaching the probe shank to a non-planar surface of a rigid shuttle for insertion of the probe shank in a brain.

In some embodiments, there is a step of permanently attaching the probe shank to a non-planar surface of a shuttle to stay in a brain.

In some embodiments, there is a step of wrapping the probe shank 360° around a shuttle to record and stimulate neurons 360° around the shuttle.

It is contemplated that any number of the individual features or steps of the above-described embodiments and of any other embodiments depicted in the drawings or description below can be combined in any combination to define an invention, except where features or steps are incompatible.

### BRIEF DESCRIPTION OF THE DRAWINGS

Preferred example embodiments will hereinafter be described in conjunction with the appended drawings, wherein like designations denote like elements, and wherein:

FIG. 1 is a schematic representation of a probe assembly, according to one embodiment;

FIG. 2 shows a probe assembly;

FIG. 3 shows a recording probe and a stimulating probe before being stacked into a probe assembly;

FIG. 4 illustrates a method of manufacturing a stimulating probe, such as the stimulating probe shown in FIG. 3;

FIG. 5 illustrates a method of manufacturing a recording probe, such as the recording probe shown in FIG. 3;

FIG. 6 illustrates a method of manufacturing the probe assembly illustrated in FIGS. 3-5;

FIG. 7 schematically illustrates a method of promoting adhesion of the polymer layers in the probe assembly of FIGS. 1-6;

FIG. 8 illustrates a method of inserting a probe assembly; and

FIG. 9 is an enlarged view of the inserted probe assembly of FIG. 8.

### DESCRIPTION OF THE ILLUSTRATED EMBODIMENTS

Described herein is a probe assembly that is an optoelectrode having a flexible probe shank to facilitate longer-term neural stimulation and monitoring. In a particular embodiment, polyimide is used (Young's modulus of less than 10 GPa while that of silicon is about 140 GPa) as a substrate material for the probe, and the probe was able to function during implantation for about one month. This embodiment of the probe assembly was a flexible optogenetic neural probe, the Blue-PARAGONS (B:PAR, standing for blue-LED-integrated Polyimide based ARtificial Apparatus for Genetically-modified Opsin in Neuron Stimulation). This particular embodiment of the probe assembly was equipped with 12 micro-ILEDs, and 32 recording electrodes all integrated in about 12 μm thick, about 115 μm wide, and 10 mm long polyimide shank, at least partially due to the microfabrication compatibility and thickness controllability of the polyimide layers that constitute the probe shank. The objective of Blue-PARAGONS (B:PAR) was to demonstrate the feasibility of a polyimide-based flexible optoelectrode integrated with micron-sized ILEDs as a chronic brain neuron cell stimulation and recording device. The device is composed of multiple individual parts, which are assembled at the end of the fabrication process and then characterized, as to accommodate multiple design requirements coming from its purposes and manufacturing processes. The multiple individual parts may include: (i) a blue-ILED integrated flexible probe for neuron cell stimulation purpose; (ii) a recording electrode probe for reading out the local field potential and action potentials from a targeted brain area of the user; (iii) an interposer: intermediate assembly component of which both probes ((i) and (ii)) are stacked and aligned, being ready for electrical connection to the backend parts of the whole assembly structure; (iv) a custom made cable; and (v) a PCB with an Omnetics connector which will work as an electrical interface to the LED driver and recording signal processing. All components are assembled into a complete flexible optoelectrode probe assembly once the fabrication is done.

There are several advantageous reasons behind the separated fabrication and assembly of the stimulation and recording probes that is described in detail herein, which is as follows: first, this allows function-based unit device manufacturing, which can increase the device fabrication yield. The overall yield of an electronic system manufacturing is equal to fabrication yield×wafer sorting yield×packaging yield, and the most significant factor that decides the yield of the flexible ILED neural probe is the wafer fabrication yield, which is defined as: wafers out of fab/wafers started in the fab. This value can be significantly impacted by any added layer of fabrication, meaning if one mask process is added. the value of wafer fabrication yield is lowered. This impact can be severe when a polyimide layer fabrication step is added, since controlling the thickness of such a polymer layer is not as easy as other materials like metal, oxide, or semiconductor due to the spin coating-based definition method that is used. The second reason is that this unit-based separated fabrication gives a freedom of choice with respect to a specific function which the end user wants. For example, an end user can choose only the stimulation function or only the recording function, and even other types of functional units such as a temperature sensor, a fluidic channel, chemical sensing or the like. This also gives the flexibility and time to an assembly function developing department since the manufacturing time is lower with less mask steps being incorporated. As long as the backend of the probes follow the standard that is being set by the design rules of the interposer, the manufacturing process can be simplified while maintaining the possibility for diverse options.

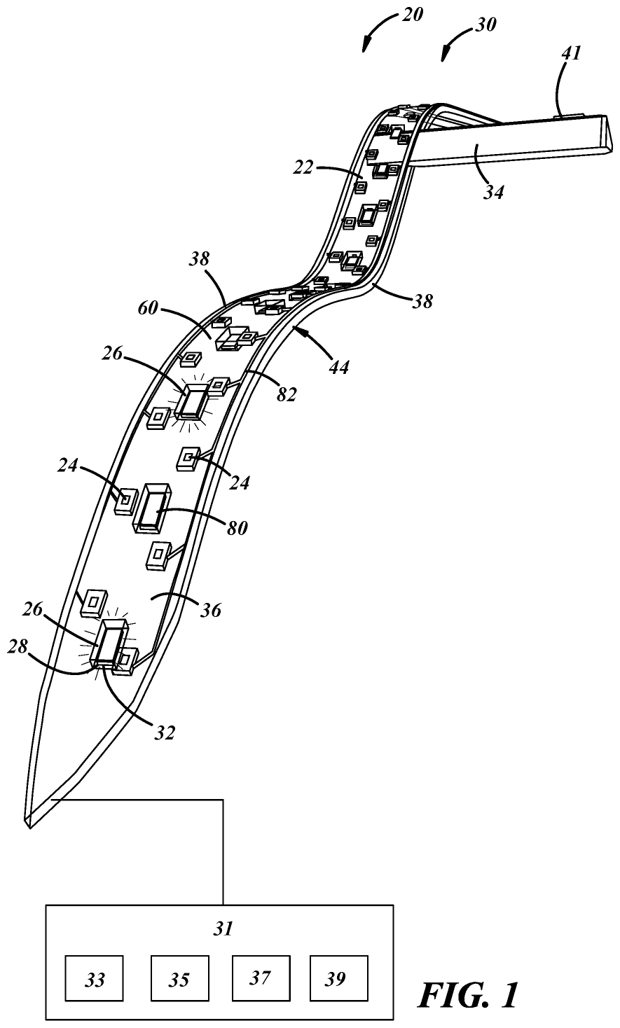

FIGS. 1-3 illustrate a probe assembly 20. With reference to FIG. 1, the probe assembly 20 includes a probe shank 22 having a plurality of recording sites 24 and a plurality of stimulating sites 26 (only a few are labeled for clarity purposes). In this embodiment, each stimulating site 26 is a light source 28, making this probe assembly 20 an optoelectrode 30. More particularly, each light source 28 in the illustrated embodiments is an inorganic light emitting diode (ILED) 32, or even more particularly, a GaN ILED, but other forms of stimulation and types of stimulating sites are certainly possible. This particular embodiment includes thirty-two recording sites 24 and twelve stimulating sites 26, but again, the number and configuration of the recording sites 24 and stimulating sites 26 can vary depending on the desired implementation. Also, the probe assembly 20 may have multiple colors of LEDs with various wavelengths. In this implementation, the probe shank 22 is about 10 mm long, which is sized to reach a target brain region, with the ILEDs 32 being spaced to stimulate individual neuron soma. A backend of the probe shank 22 is coupled to an interposer 34, which can ultimately be coupled to a printed circuit board (PCB), which can be operated through a wire connected connector, such as an Omnetics connector.

The thickness of each ILED 32 is about 2.5 μm, and the pitch from one LED 32 to another is 83 μm. The LEDs 32 are designed to be placed in the middle side of the shank 22 for them to function as a centered blue light (λpeak≈467 nm) illumination source so the light power can be evenly delivered to a top surface 36 of the probe located cell (blue light induces the activation of genetically modified channelrhodopsin-2) while the recording sites 24 are placed on the side of the LEDs 32, sensing the potential variation of the neuron membrane surrounding environment from multiple sites. Twelve LEDs 32 are integrated in the middle of a 6 μm thick polyimide probe shank 22 and connected to the backend of the LED probe assembly 20 with 300 nm thick metal traces, total 10 mm long and electrically connected to the PCB through the interposer 34 (ball bonded) and a custom-made flexible cable (e.g., about 2 cm long). Other types of light sources 28 include organic LEDs (OLEDs) 32, quantum dots (QD), or another electroluminescent device.

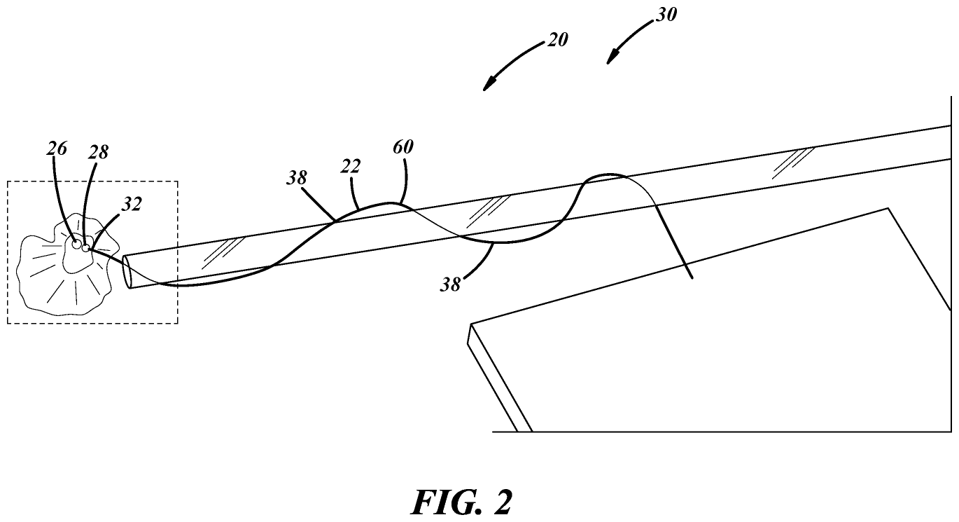

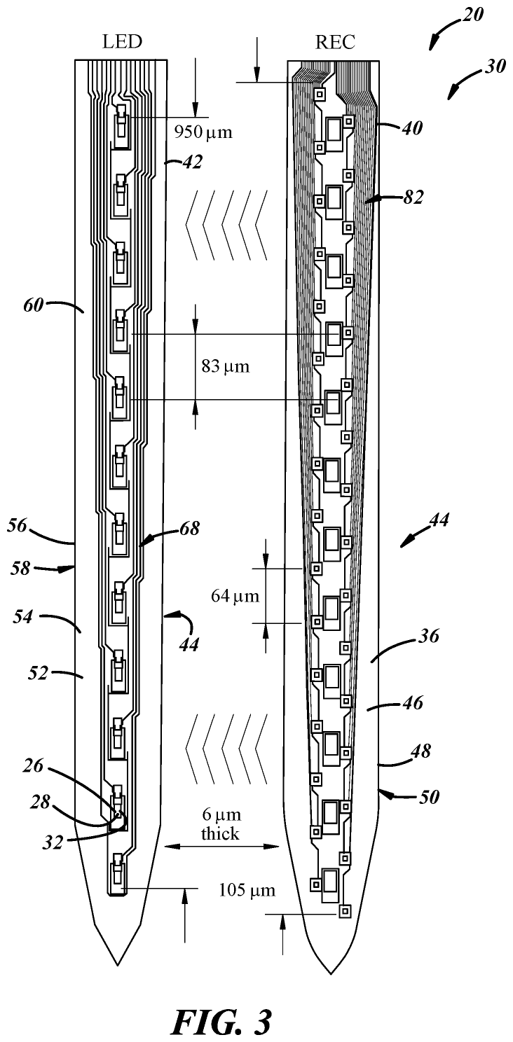

To achieve long-term stimulation and monitoring, as opposed to more rigid or semi-rigid probe shanks, the probe shank 22 is flexible and configured to conform around an anatomical structure. As shown in FIGS. 1 and 2, the entirety of the shank 22 can twist and flexibly bend to help facilitate long-term implantation (e.g., a month or more), as opposed to silicon shanks, for example, which are more typical yet have the potential to damage tissue if implanted for a longer period of time. The probe shank 22 accordingly has multiple curvilinear segments 38, with both recording sites 24 and stimulating sites 26 being located along each curvilinear segment 38. FIG. 1 also schematically shows functional subassemblies 31 that can be used in the probe assembly 20, which include but are not limited to a metal shielding layer 33 located between the recording probe 40 and the stimulating probe 42 (see also FIG. 3), temperature sensors 35, neurotransmitter sensors 37, and microfluidic channels or layers 39 which can be used to introduce drugs and/or reagent for neurostimulation. FIG. 1 also shows a custom circuit chip 41 which is hybrid-integrated in the interposer 34 to change and enhance the signal-to-noise ratio of recording signals and reduce the number of traces by digitally multiplexing the recording and stimulation signals.

With reference to FIG. 3, the probe assembly 20 is constructed by separately manufacturing a recording probe 40 and a stimulating probe 42, and then stacking the recording probe and the stimulating probe together to form the final assembly. Each probe 40, 42 comprises a plurality of polymer layers 44 to impart the conformal structure and requisite flexibility needed to form the multiple curvilinear segments 38. The plurality of polymer layers 44 includes a top surface polymer layer 46 on the top surface 36 of the recording probe 40, an interface polymer layer 48 on the bottom surface 50 of the recording probe, an interface polymer layer 52 on the top surface 54 of the stimulating probe 42, and a bottom surface polymer layer 56 on the bottom surface 58 of the stimulating probe and the probe shank 22. As detailed further below, there may be more polymer layers than what is shown, or there could be less. For example, if the probe assembly 20 only serves to record instead of stimulate, the probe shank 22 may only comprise two polymer layers 46, 48 in the plurality of polymer layers 44.

Each polymer layer of the plurality of polymer layers 44 is made from a polymer-based material. To impart the requisite flexibility, in one embodiment, the polymer-based material has a Young's modulus of 10 GPa or less. This is magnitudes less than semi-rigid or rigid materials, such as silicon, which has a Young's modulus of about 140 GPa. In an advantageous embodiment, the polymer-based material is polyimide-based, or a mixture containing 50 wt % or more polyimide. Using polyimide for the entirety of the probe shank 22 and each of the layers of the plurality of polymer layers 44 helps impart the conformal nature of the curvilinear segments 38 (i.e., 90-100 wt % polyimide). In one particular implementation, the polymer layers 44 are manufactured from polyimide 2610 and 2611 that are separately spun and cured to create sublayers of varying polyimide types within each polymer layer. Polyimide 2610 and 2611 may be easier to work with than other polyimide types, viscosities, etc. Moreover, this polyimide-based structure for the layers 44 and probe shank 22 allows for the formation of a partially transparent body 60. The partially transparent body 60 in the illustrated embodiment is configured to allow about 70-80% of blue light emission from each light source 28 (wavelength 400-500 nm, with 467 nm peak wavelength). This amount of emission provides enough blue light to the cells placed outside of the shank 22 for adequate neural stimulation. In another embodiment, one or more Kapton layers are used for the plurality of polymer layers 44. Other polymer-based materials for the polymer layers 44 are possible, such as parylene, PDMS, or silicone to cite a few examples.

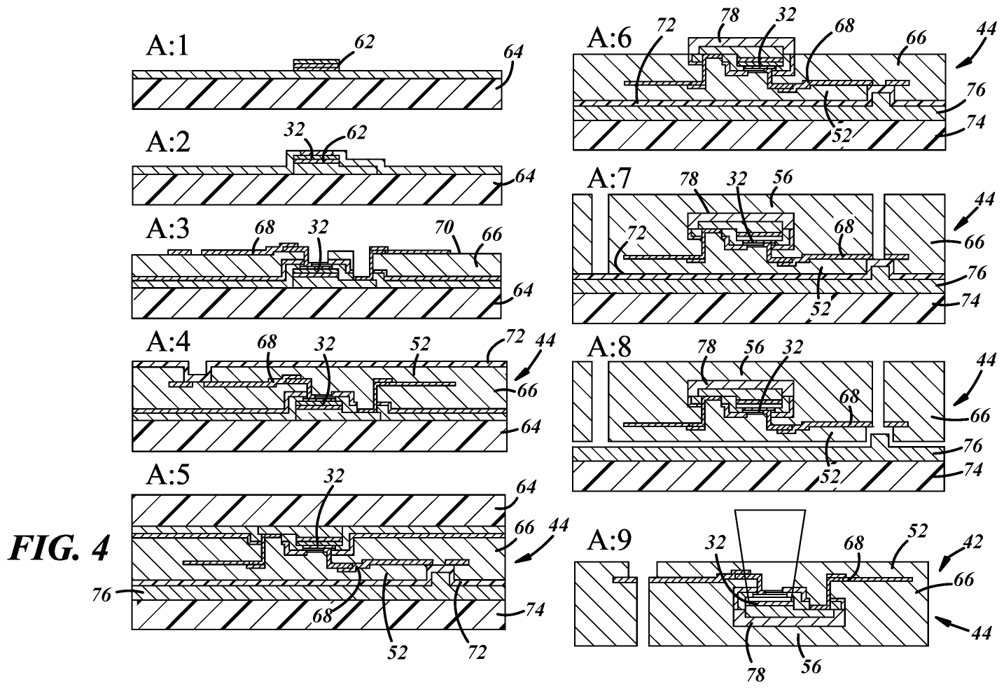

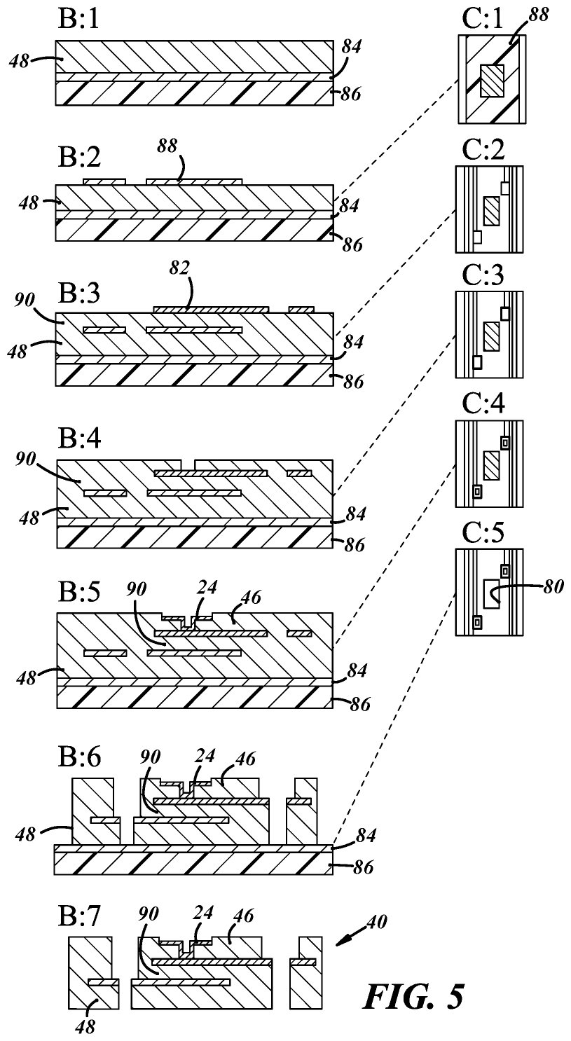

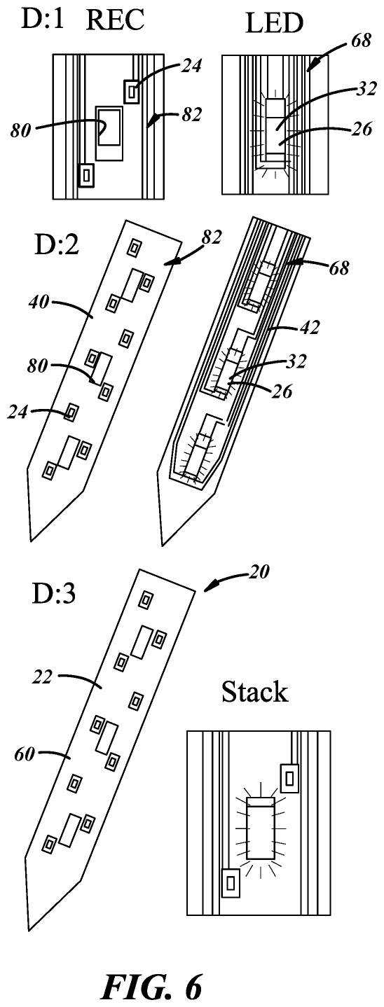

FIGS. 4-7 schematically illustrate the manufacture of the probe assembly 20, with FIG. 4 showing manufacture of the stimulating probe 42, FIG. 5 showing manufacture of the recording probe 40, and FIG. 6 illustrating the stacking of the recording probe 40 and the stimulating probe 42. It should be understood, however, that these figures and the description herein describe only one method of manufacture, and variations thereto are certainly possible. For example, the probe assembly 20 could be manufactured with more or less polymer layers, or with different configurations for the recording sites 24 and/or stimulating sites 26.

With reference to FIG. 4, at step A:1, the fabrication process of the probe assembly 20 starts with a GaN-based multiple quantum well (MQW) 62 grown on a silicon (111) wafer 64 (e.g., about 4 inch). The mesa structure of the LED 32 can be defined by two steps, one the first mesa etching down to the n-contact region of the LED (e.g., about 500 nm), second the etching down to the field silicon region (e.g., about 2 μm, total mesa stack height of about 2.5 μm). The silicon region 64 should be exposed for later processing, especially for the full-coverage of the LEDs 32 by the polyimide layer 52. Cl₂ and BCl₃ mixture gas can be used for the RIE definition of the mesa. Once the mesa has been formed, the resist is peeled off and the uniformity check (e.g., 9-point measurement in 4″ wafer) can be done, which gave less than 5% variation of the total mesa height among the whole wafer. If the variation is severe, or greater than 5%, the uniformity of the thickness of the polyimide deposited later can degrade which then can be problematic during the wafer transferring process, metal trace definition, and LED backside processing including silicon removal, etc. The LED 32 is preferably a micro-LED (e.g., less than 500 square microns, or preferably less than 100 square microns) that is monolithically fabricated.

In steps A:2 and A:3, the prepared bare LED 32 mesa wafer is then passivated with a thin oxide layer (e.g., about 500 nm) for the purpose of mesa passivation itself from the post processing of wet etching etc., and to disunite the bottom silicon substrate 64 from an intermediate polyimide layer 66. This separation is beneficial in terms of blocking the bottom of the polyimide layer 66 from the direct exposure to the DRIE plasma during silicon 64 removal. The p-GaN metal contact is defined using 4.3 μm thick SPR 220 3.0 photoresist lithography. After development, the Ni/Au (e.g., total 10 nm thick) metal stack is evaporated and lifted-off, and the wafer is then treated with 500° C. rapid thermal annealing (RTA) for lowering contact resistance and transparency of the metal contact. The contact holes for n-GaN can be opened through reactive ion etching (RIE) and wet etching, and the wafer 64 is once again passivated with Al₂O₃. On top of the oxide deposited wafer 64 the intermediate polymer layer 66 is spun and cured at 350° C. inside the oven using an H₂/N₂ environment (e.g., thickness of about 1.5 μm after cured). This layer 66 advantageously consists of polyimide 2610. Once the polyimide layer 66 is defined, the contact holes are etched through oxygen plasma etching, and treated with wet buffered HF etching to remove the remaining thin oxide layer. Then the n-GaN contact metal and p-GaN contact metal to trace connection layer is defined again using the double-layer photoresist lithography utilizing LOR. This consists of the thin LOR and S1813 resist, a total of about 3 μm thick. This double-layered undercut structure can be beneficial in achieving sputtering and lift-off. After development is done, the Cr and Au layers (total 300 nm) are sputtered and lifted-off. Finally, the LED p-GaN contact metal/intermediate metal and n-GaN contact metal is wired to the backend ball bonding part of the probe through 300 nm thick stimulating traces 68, which are composed of Ti and Au in this embodiment. The stimulating traces 68 in this embodiment are electrical leads used for the LEDs 32, but in other embodiments, the stimulating traces 68 may or may not be standard electrical traces, as they may facilitate one or more other types of cell stimulation (e.g., thermal, chemical, etc.).

In steps A:4 and A:5, once traces 68 for the LEDs 32 were completed, a layer 52 of polyimide is again spun and cured (e.g., about 2.5 μm). As detailed further below, an adhesion portion 70 of the first/intermediate polyimide layer 66 is treated with oxygen plasma for better adhesion between polyimide interfaces (PI-to-PI). Then, the bonding pads at the backend of the LED stimulating shank 42 are opened for ball bonding, and a sacrificial layer 72 (e.g., chromium, total 220 nm) is then sputtered (Lab 18-02) on the whole wafer surface and the wafer is now ready to be transferred. To transfer the LED wafer 64 to the other carrier wafer 74 for the purpose of a complete removal of the rigid silicon substrate 64, a special adhesive 76 is used that can preferably (i) withstand the 350° C. one hour of curing process and (ii) last without being etched or delaminated during acid (HF) and chromium wet etching. The material adopted for the adhesive layer 76 is advantageously HD-3007, which is a polyimide-based adhesive. Because the adhesive 76 is a polyimide-based material and its curing process is similar to polyimide, it can withstand the curing process without being etched or delaminated during subsequent processing steps. On the top of the LED wafer and the carrier wafer (10E15 Boron doped silicon wafer with 550 μm thickness) the adhesive 76 is spun and cured (about 3 μm thick for each, cured under N₂ environment), and bonded (the top-surfaces) using the wafer bonding equipment, under vacuum at 350° C. The flatness (including the uniformity of the mesa height) of the LED wafer can be critical during this step for uniform bonding (if not, the voids inside can hinder the backside processing and lower the die yield).

In step A:6, the bonded wafer is ready for backside silicon 64 removal through a deep-reactive-ion-etching (DRIE) step. Since the intermediate space between one silicon wafer 64 to the other wafer 74 is composed of low-thermally conductive layers 66, 52 (ultimately a total of 3 layers, thickness of about 10 μm, the thermal conductivity of polyimide is 0.12 W/m·K while that of silicon is 148 W/m·K), and since the DRIE process brings heat into the plasma exposed substrate, the etching step should be carefully executed. Pure etching duration was 6 hours (e.g., etch rate of about 2.2 μm/min). Another method that may be used is to first CMP (chemical mechanical polishing) the silicon substrate and thin down until about 100 μm thick is remaining, and finish etching through DRIE for reduced substrate removal duration. The silicon 64 is completely removed, and the field oxide is now wet etched away with HF solution. An exposed bottom surface of the n-GaN of LED mesa 32 is covered by titanium and aluminum which also serves as the probe backside light blocking layer 78.

In steps A:7 through A:9, the third layer of polyimide 56 is spun and cured, sandwiching and surrounding the LEDs 32 and traces 68 all around with the flexible polyimide shank 22 (e.g., about 2 μm thick, about 6 μm of total LED probe thickness). The through-holes for the ball bonding pads are opened through oxygen plasma RIE and at the same time probe outline is defined. Then the wafer can be put inside of a chromium etchant for probe release. Once the probes 42 are released, they are moved to DI water and stored.

FIG. 5 schematically illustrates manufacture of the recording probe 40, which, in this embodiment, is composed of thirty-two electrodes 24 that are arranged as two rows inside the 115 μm wide flexible polyimide probe shank 22, with the stimulation probe 42 that is subsequently stacked against the bottom side of it. Advantageous conditions for a flexible recording electrode probe 40 are as follows: the impedance of the fully assembled device should show less or similar to 2 MΩ (at 1 kHz of measurement setting), the probe shank 22 size (especially the width) should be minimized so that the tissue damage during surgery and reaction to it is reduced, and an optical window 80 (an LED-sized opened area) should be formed for effective LED 32 light delivery to the targeted area. For each requirement the recording probe 40 was designed to have thirty-two recording traces 82 and each has 1 μm width and 1 μm distance from one another, so that all the traces 82 and electrodes 24 can be placed inside the 115 μm wide probe shank 22, and the surface of the electrodes 24 are roughened to increase the effective area of it (then the impedance is lowered accordingly). The LED optical window 80 was designed to have minimum 3.5 μm alignment margin from the edge of the LED 32 (this was defined taking consideration on margin for the fabrication using stepper and the difference between recording probe 40 and LED probe 42 width that may occur during probe outline definition). Advantageously, the recording probe 40 is stacked on top of the LED probe 42, instead of being the other way around, allowing the recording electrodes or sites 24 to be placed as close as possible to the neuron soma inside the brain tissue. The area of each recording electrode 24 was designed as 195 μm², and the vertical and lateral distance of the electrodes were set to 17 and 22 μm so that the diagonal distance between two closest electrodes would be about 30 μm, while the vertical distance between two electrodes at the same column is designed to be 64 μm, which gives 1000 μm of covering span from top to bottom.

The fabrication process flow is summarized and represented as schematics in FIG. 5, steps B:1 through B:7, and C:1 through C:5. Manufacturing the recording probe 40 starts with a bare thin oxide grown silicon wafer 86. First, the chromium sacrificial layer 84 (e.g., about 225 nm) is defined first (not like the LED probe 42 since the recording probe 40 fabrication does not incorporate a wafer transferring process). Then, the first polyimide layer 48 is spun and cured (e.g., about 2 μm) followed by the bottom metal shield 88 definition in step B:2, which works as the electrical shield blocking the electromagnetic interference (EMI) generated in LED probe traces 68. The shield 88 is composed of titanium and gold (e.g., about 300 nm) and is ball bonded to the ground at the end of the assembly process. Step C:1 shows the top view of defined shield 88 with the LED optical window 80 patterned.

In steps B:3 and B:4, along with C:2 and C:3, on the top of the defined bottom-shield layer 88, the second or intermediate polyimide layer 90 is spun and cured (e.g., about 1.8 μm thick) as the intermediate layer between the shield 88 and the second metal for the traces 82 for connecting recording electrodes 24 and the backend ball bonding pads. For the purpose of enhancing the passivation of the recording traces 82 inside the brain tissue, especially on the top side which is closer to the top-most surface of the recording probe 40 with only one layer 46 of polyimide passivating it, Al₂O₃ was chosen to be the material to surround the trace 82 for its promising water vapor transmission rate (WVTR). About 15 nm of Al₂O₃ is first deposited through atomic layer deposition and then the 2.5 μm thick photoresist is patterned and developed for the trace shape, and again titanium and gold metal composites are evaporated and lifted off. Once again, the top side of the traces were covered with additional passivation layer (e.g., about 15 nm) and the oxide is patterned, leaving the probe-outline boundary free from any oxide coverings. Next, the third polyimide layer 46 is spun and cured (e.g., about 2 μm thick) and the recording trace-to-recording electrode connection holes are etched away, having the wafer being ready for electrode fabrication.

With reference to steps B:5 through B:7 and C:4 through C:5, to decrease the impedance as much as it can be, an electrode surface roughening technology was adopted. Titanium islands were sputtered and the polyimide 46 was etched to form the hills and valleys, described further below, throughout the electrode 24 surface area, followed by electrode metal sputtering (e.g., titanium and platinum, about 100 nm) and lift-off. Then the recording probe outline lithography and RIE was processed, and the probe 40 was released inside the chromium etchant.

As shown schematically in FIG. 6, once the recording probe 40 and the stimulating probe 42 are constructed, the two probes can be stacked to form the probe assembly 20. The LEDs 32 of the stimulating probe 42 are advantageously aligned with each optical window 80 in the recording probe 40. This results in a probe shank 22 having the LEDs 32 being placed in the mid-space to illuminate from the middle of the two columns of recording sites 24. The optical window 80 allows for only a single polymer layer 52 to be coving the LED 32, which can help achieve desirable light emission levels. As described above, the recording probe 40 is stacked on top of the stimulating probe 42, preferably after being treated with IPA, and the stacked probe shank part 22 is dipped into the DI water for finer alignment. The backend of the stacked probe assembly 20 was ball-bonded to the interposer 34 first, and passivated. Then, the cable is ball bonded to the interposer 34 and the PCB, and finally the connectors are attached to the PCB through wires or directly through thermal reflow of solder.

The flexible probe shank 22 is advantageously used for chronic experimentation, which means the probe assembly 20 can stay inside the brain for a long time. Additionally, the probe assembly 20 can be designed to be inserted into a specific region inside the brain. Also, it should have a structure that is easy to handle during surgery, that is to say, it is better to have the backend of the modules as light-weighted and long as technically feasible. Here, a thin silicon-based interposer structure 34 was chosen as an intermediate assembly part to merge the modules, as its processing is straightforward and the density of the metal trace inside it can be easily increased. Compared to having long-back-ended modules and directly bonding them to the PCB, interposer 34 allows a much higher yield (the number of total functioning probe assemblies 20 fabricated for a unit time increases, since the number of the module probes 40, 42 that can be fabricated inside one wafer is raised) since the processing time, especially the LED probe 42, is long. The interposer 34 is composed of the bonding pads on its front-end side for probe modules alignment and bonding, and on its back-end side the pads for connecting the cable is designed.

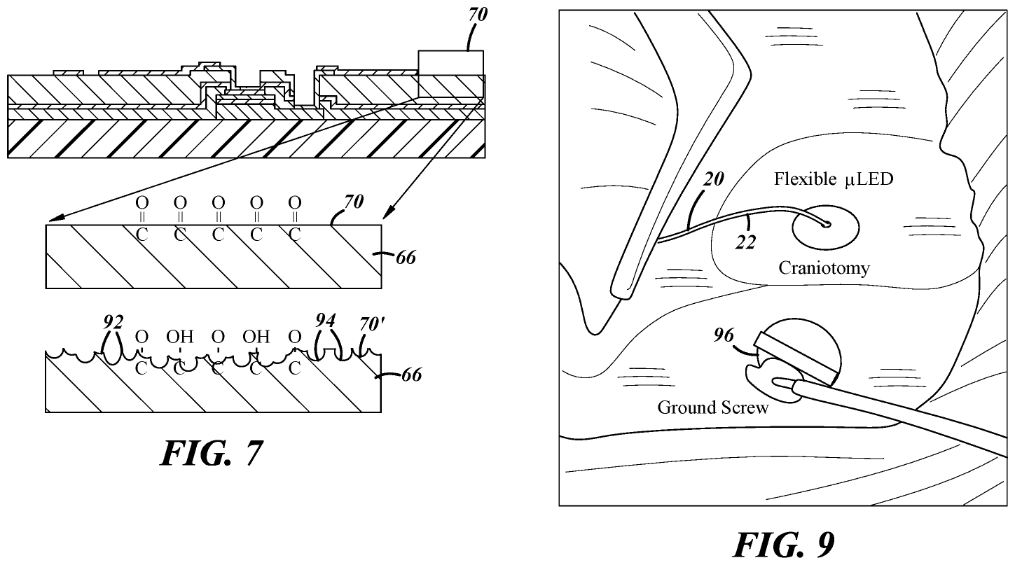

FIG. 7 shows a schematic representation of the oxygen plasma treatment that can be used to enhance adhesion at polymer-polymer interfaces. The adhesion portion 70 represents the polyimide layer 66 after spin and curing. The adhesion portion 70′ is after the oxygen plasma treatment, which creates a plurality of hills 92 and valleys 94 having C—O or C—OH hydrophilic chemical bonds along the adhesion portion 70′. This can increase the clean surface area available for bonding, along with increasing the hydrophilic chemical characteristics of the adhesion portion 70′. As shown, as opposed to double bonded oxygen atoms, there are more single bonds, with increased C—O and C—OH bonding on the adhesion portion 70′. This can enhance the connection between the plurality of polymer layers 44. This arrangement can also be particularly beneficial when an adhesion portion and the hills and valleys of one adhesion portion 70′ on the polymer layer 66 interface against the adhesion portion 70′ and the hills and valleys of another polymer layer in the plurality of polymer layers 44.

During testing of the probe assembly 20, about 80% of the LEDs 32 had a current value between about 11 μA and 15 μA (note that the Vin used during in-vivo testing was 3.8 V). Then, the wavelength spectrum of the blue-light generated from the LEDs were tested for various current levels. The peak amplitude was measured to be ranging at 466-468 nm (467 nm at 15 μA of testing current). The radiant flux (Φₑ) was also measured for 15 and 50 μA of current levels. The median value points were about 1.5 and 4.5 μW for 15 and 50 μA respectively. Considering the lower threshold value (optical power needed) that is required for the opsin stimulation is 1 mW/mm², the optical power measured from the probe assembly 20 was suitable as a stimulation functioning probe.

Following the electrical and optical characterization, the impedance of the recording electrodes 24 was tested in-vitro. More than 90% of the electrodes 24 showed the impedance value between about 1 and 1.5 MΩ, which is suitable to be used for recording of the variation of the local field potential inside the brain target region. The impedance value was then further tracked during the in-vivo testing. The feasibility of the device in terms of the heating effect should also be evaluated, which can be important in preventing severe heating and tissue degeneration of the brain. COMSOL Multiphysics was used for the simulation of the brain tissue heating and temperature analysis, and the very same dimensions of the LED probe were used in the analysis. The 6 μm×15 μm area for each LED 32 was designated as the light power generating area, and the LED was covered with a polyimide layer, with thermal conductivity of 0.12 W/m·K, compared to a more typical, rigid silicon shank probe (with the same dimension, total thickness of 12 μm) with a much higher thermal conductivity of 148 W/m·K. The probing point of the temperature was set to be the 8 μm z-axis direction higher point from where the LED top surface is at, as this is the actual height where the cell can sit practically. The thickness of the polyimide layer 52 on the top of the LED 32 is about 2.5 μm, and the thickness of the recording probe 40 is about 6 μm, and the summed-up value gives at least 8 μm as an upper point where there will be a vacancy where a cell can sit. The input power signal was set as at 1 sec long square function with 10% duty cycle. The power was varied from minimum 37 μW to 105 μW where the values were chosen from the actual power range at the input voltage range of 3.0 V to 4.5 V, while having the power input criteria as 60 μW (this was set as the point of Vin=3.8 V, I=16 μA) for the comparison of the polyimide-based and silicon-based simulation results. Even with continued cycling of the input power, the peak temperature value did not increase, and as soon as the input pulse became off, the temperature would also rapidly go down and saturate to the set baseline temperature (which is 37° C.). Even with the 105 μW of input power, the difference between temperature peak value and the baseline is less than 1° C., which is beneficial in an in-vivo study. While the temperature variation is less with a silicon-based probe, the polyimide-based flexible probe was also shown to be suitable.

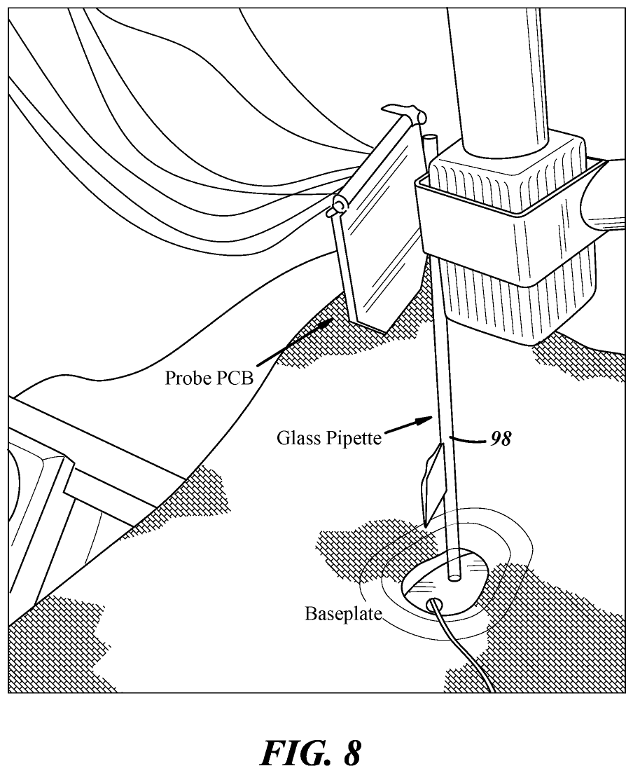

FIGS. 8 and 9 show implantation of a probe assembly 20. FIG. 8 shows the surgery process, and FIG. 9 shows the inserted flexible LED probe shank 22. An adult male mouse (CaMKII-ChR2, 31 g) was kept in a vivarium on a 12-hour light/dark cycle and was housed two per cage before surgery and individually after it. Atropine (0.05 mg kg-1, s.c.) was administered after isoflurane anesthesia induction to reduce saliva production. The body temperature was monitored and kept constant at 36-37° C. with a DC temperature controller. Stages of anesthesia were maintained by confirming the lack of a nociceptive reflex. The skin of the head was shaved, and the surface of the skull was cleaned by hydrogen peroxide (2%). A custom 3D-printed base plate was attached to the skull using dental cement. A stainless-steel ground screw 96, as shown in FIG. 9, was placed above the cerebellum and sealed with dental cement. The location of the craniotomy was marked (2 mm posterior to Bregma and 1.5 mm lateral to midline) and a 700-μm craniotomy was drilled. After the dura was removed the flexible probe shank 22 was inserted into the brain (1.5 mm depth from the surface of the brain) using a glass pipette 98 with a tip diameter of 15-20 μm, as shown in FIG. 8. The glass pipette 98 was retracted, and the craniotomy was sealed with dura-gel and then covered with dental cement. Finally, a protective cap was built using copper mesh. The mouse recovered for at least 7 days after surgery. The animal was recorded in its home cage. The collected data was digitized at 20 kS/s using an RHD2000 recording system.

The glass pipette 98 was used as the shuttle because a rigid shuttle is needed for the surgery since the flexible probe shank 22 is not strong enough to penetrate the tissue itself. The probe tip was attached to the glass pipette 98 using polyethylene glycol. Once attached, the shuttle 98 is then lowered to the brain surface and penetrates, and the probe shank 22 is placed inside the hippocampal region of the mouse brain. The glass pipette 98 was retracted after few hours (after the probe shank 22 and the pipette 98 detach from each other), leaving the flexible probe assembly 20 only inside the hippocampus. The probe shank 22 may be temporarily placed on a non-planar surface of the shuttle 98 for insertion and then detached when retracting the shuttle. In other embodiments, the probe shank 22 may be permanently attached to the non-planar shuttle 98 and stay inside the brain. In one implementation, the probe shank 22 can be wrapped around the non-planar surface (e.g. a hexagonal, octagonal, or circular cross-sectional shape for the shuttle 98) to record and stimulate neurons 360° around the shuttle 98. After surgery, the mouse was awakened from the anesthesia with the probe assembly 20 fixed on the top of its head, and waited for two weeks so that the tissue would be healed near where the probe shank 22 was embedded. The interposer 34 is connected between the probe shank 22 and the cable which gives extended flexibility of the entire probe assembly 20 to minimize micromotion of the headstage.

For the in-vivo validation, first after 2 weeks of surgery the cell classification was performed and a total of seven isolated cells were discovered and the waveforms from four putative pyramidal and three putative narrow waveform interneurons were recorded simultaneously. It was demonstrated that the embedded probe shank 22 (or more particularly, the recording electrodes 24) were working properly for the purpose of observing the spikes of the neurons. Based on the recorded signals, the third LED 32 (when the tip-most is the #1 and the backend-most is #12) was chose to be lit to test the illumination and stimulation-induced spike generation.

For stimulation testing, a 1 sec long Vin with the duty cycle of 10% was used and illuminated light power was 1 μW. LED-light induced spiking around the LEDs 32 was shown, along with recorded neural signal waveforms (putative pyramidal cell). The cell was stimulated by the LED 32 significantly and more than ten times of difference in spiking rate was observed. The same experiment was held at day 18 and day 26, and the same LED (third one from the tip-most side of the probe shank 22) was used for stimulation for both of the repeated experiment, with the same amount of Vin (amplitude of 3.8 V, 1 sec long pulse with 10% duty cycle) was used. The putative pyramidal cell spike was strongly induced by the blue-LED illuminated 467 nm of wavelength of light. The probe assembly 20 functioned as the stimulation/recording optoelectrode 30 inside the brain chronically for about 4 weeks of total experiment time duration. Note that to determine the recording electrodes 24 were not shorted or opened during experiment, the impedance of them was tracked and represented.

Chronic in-vivo experiment revealed the capacity of the probe assembly 20 as a fully-functioning, flexible, chronic application through following results: first, the objective of the recording electrode probe 40 was proven through the cell classification (total seven near putative pyramidal cells and interneurons) at the second week; and secondly, the optogenetic objective of the LED probe 42, blue-light (467 nm peak wavelength) from ILED 32 illumination-induced spikes were recorded and compared with non-illuminating state and revealed the stimulation-induced spiking rates are increased significantly. Lastly, the chronic-capability, was completely validated through the in-vivo experiment at the fourth week from the surgery, proving the spiking rate discrepancy between non-illuminating and illuminating states showed similar trend to that of the second week.

It is to be understood that the foregoing description is of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “e.g.,” “for instance,” and “such as,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation. In addition, the term “and/or” is to be construed as an inclusive OR. Therefore, for example, the phrase “A, B, and/or C” is to be interpreted as covering all the following: “A”; “B”; “C”; “A and B”; “A and C”; “B and C”; and “A, B, and C.”

1. A probe assembly, comprising:
a probe shank comprising a plurality of polymer layers, including a first polymer layer and a second polymer layer;
one or more recording traces; and
one or more stimulating traces, wherein the first polymer layer and the second polymer layer sandwich the one or more recording traces or the one or more stimulating traces such that the first polymer layer, the second polymer layer, the one or more recording traces, and the one or more stimulating traces are configured to conform around an anatomical structure.

2. The probe assembly of claim 1, wherein the plurality of polymer layers is made from a polyimide-based material.

3. The probe assembly of claim 1, wherein the plurality of polymer layers is made from a parylene-based material, a PDMS-based material, or a silicone-based material.

4. The probe assembly of claim 1, wherein one or more polymer layers of the plurality of polymer layers includes a plurality of hills and valleys.

5. The probe assembly of claim 1, wherein the probe shank comprises a stimulating probe having the one or more stimulating traces stacked against a recording probe having the one or more recording traces.

6. A probe assembly, comprising:
a recording probe having one or more recording traces located between a plurality of polymer layers; and
a stimulating probe having one or more stimulating traces located between a plurality of polymer layers, wherein the stimulating probe is stacked against the recording probe.

7. The probe assembly of claim 6, wherein the recording probe includes an optical window configured to at least partially expose a light source on the stimulating probe.

8. The probe assembly of claim 7, wherein the light source on the stimulating probe is an inorganic light emitting device (ILED), an organic light emitting device (OLED), a quantum dot (QD), or an electroluminescent device.

9. The probe assembly of claim 7, comprising a plurality of light sources, wherein one or more light sources of the plurality of light sources are different colors having different wavelengths.

10. The probe assembly of claim 6, wherein a metal shielding layer is placed between the recording probe and the stimulating probe.

11. The probe assembly of claim 6, wherein various functional subassemblies are stacked together, the various functional assemblies including one or more temperature sensors, one or more neurotransmitter sensors, and/or one or more microfluidic layers or channels.

12. The probe assembly of claim 6, wherein an interposer is connected between a cable and the recording probe and the stimulating probe to minimize micromotion of a headstage.

13. The probe assembly of claim 12, wherein a circuit chip is hybrid-integrated in the interposer to change the signal-to-noise ratio of recorded signals and reduce a number of traces by digitally multiplexing stimulation signals and the recording signals.

14. A method of manufacturing a probe assembly, comprising the steps of:
treating a first polymer layer to increase a surface area on an adhesion portion of
the first polymer layer; and
adhering the first polymer layer to a second polymer layer, wherein the adhesion portion of the first polymer layer contacts the second polymer layer.

15. A method of manufacturing a probe assembly, comprising the steps of:
monolithically fabricating a light emitting diode (LED) that is equal to or less than 500 square microns; and
applying a polyimide layer to form a probe shank around the LED.

16. The method of manufacturing of claim 15, comprising the step of temporarily attaching the probe shank to a non-planar surface of a rigid shuttle for insertion of the probe shank in a brain.

17. The method of manufacturing of claim 15, comprising the step of permanently attaching the probe shank to a non-planar surface of a shuttle to stay in a brain.

18. The method of manufacturing of claim 15, comprising the step of wrapping the probe shank 360° around a shuttle to record and stimulate neurons 360° around the shuttle.
